(19) United States
(12) Patent Application Publication
(10) Pub. No.: US 2011/0245632 A1
(43) Pub. Date: Oct. 6, 2011

(54) Medical Diagnosis Using Biometric Sensor Protocols Based on Medical Examination Attributes and Monitored Data

(75) Inventors: Sailesh Chutani, Redmond, WA (US); David M. Zar, Maryland Heights, MO (US); Nikhil J. George, Redmond, WA (US)

(73) Assignee: MobiSante Inc., Redmond, WA (US)

(21) Appl. No.: 12/938,338

(22) Filed: Nov. 2, 2010

Related U.S. Application Data
(60) Provisional application No. 61/400,709, filed on Aug. 2, 2010.
(60) Provisional application No. 61/341,734, filed on Apr. 5, 2010.

(51) Int. Cl. A61B 5/00

(52) U.S. Cl. 600/301

(57) ABSTRACT

A method comprises obtaining, by a computer, patient information associated with a patient and operator information associated with one or more biometric sensors; determining, by the computer, a protocol for obtaining patient data from the patient based on the patient information and the operator information; obtaining, by the one or more biometric sensors, the patient data from the patient based on the protocol.

### CROSS-REFERENCE TO RELATED APPLICATIONS; BENEFIT CLAIM

This application claims the benefit under 35 U.S.C. §119(e) of Provisional Appln. 61/400,709, filed Aug. 2, 2010, the entire content of which is hereby incorporated by reference as if fully set forth herein.

This application claims the benefit under 35 U.S.C. §119(e) of Provisional Appln. 61/341,734, filed Apr. 5, 2010, the entire content of which is hereby incorporated by reference as if fully set forth herein.

### TECHNICAL FIELD

The present disclosure generally relates to determining a protocol for performing a medical diagnosis in a computer coupled to a biometric sensor. The disclosure relates more specifically to determining a protocol for a medical diagnosis based on medical examination attributes or monitored data.

### BACKGROUND

The approaches described in this section could be pursued, but are not necessarily approaches that have been previously conceived or pursued. Therefore, unless otherwise indicated herein, the approaches described in this section are not prior art to the claims in this application and are not admitted to be prior art by inclusion in this section.

The advent of medical diagnostic devices has changed the manner in which medical personnel collect and evaluate patient data. Medical diagnostic devices include biometric sensors such as ultrasound probes which can obtain patient data for visualizing subcutaneous body structures including tendons, muscles, joints, vessels and internal organs for possible pathology or lesions. For example, obstetric sonography, which is commonly used during pregnancy may be used to visualize a fetus.

Traditionally medical diagnostic devices have been large in size and stationed in particular rooms within a hospital setting or medical office. Recently, portable medical diagnosis devices have been developed for obtaining data from patients in their homes, medical offices, or other suitable locations. The portable medical diagnosis devices are generally lower in costs and are more accessible for patients.

### BRIEF DESCRIPTION OF THE DRAWINGS

In the drawings:

FIG. 1A illustrates a computer system in accordance with an embodiment;

FIG. 1B illustrates an example of biometric sensor management logic in accordance with an embodiment;

FIG. 2A and FIG. 2B illustrate determining a protocol in accordance with an embodiment;

FIG. 3 illustrates an example of a biometric sensor in accordance with an embodiment;

FIG. 4 and FIG. 5 illustrate examples of one or more computers upon which one or more embodiments may be implemented.

### DETAILED DESCRIPTION OF ONE OR MORE EXAMPLE EMBODIMENTS

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

### General Overview

In an embodiment, a method comprises: obtaining, by a computer, patient information associated with a patient and operator information associated with one or more biometric sensors; determining, by the computer, a protocol for obtaining patient data from the patient based on the patient information and the operator information; obtaining, by the one or more biometric sensors, the patient data from the patient based on the protocol.

The protocol may include one or more of: an ultrasound sensor probing sequence; a configuration setting for the one or more biometric sensors, or positioning information for the one or more biometric sensors.

In an embodiment, determining the protocol is further based on exam type information associated with a medical exam that could be performed on the patient to obtain the patient data.

In an embodiment, the method further comprises determining, by the computer, a subgroup of one or more protocols from a plurality of protocols for obtaining patient data from the patient based on the patient information and the operator information; and presenting the subgroup of one or more protocols as options for use with the one or more biometric sensors.

In an embodiment, the method further comprises comparing, by the computer, a first patient data that is newly obtained by the one or more biometric sensors with a second patient data that was previously obtained by the one or more biometric sensors and stored on the computer, wherein the computer is communicatively coupled with the one or more biometric sensors.

In an embodiment, a method comprises monitoring, by a computer, a biometric sensor obtaining first patient data from a first patient during a medical examination; recording monitoring data comprising one or more configurations, of the biometric sensor, while the first patient data was being obtained; generating a protocol for obtaining second patient data from a second patient based on the monitoring data; wherein the method is performed by at least one computer.

The one or more configurations may comprise: a plurality of positions for the biometric sensor while the first patient data was being obtained; a test sequence for obtaining the first patient data; or device settings for the biometric sensor.

In other aspects, the disclosure encompasses an apparatus with means for the functionality described herein and a computer readable medium comprising instructions, which when executed by one or more processors provide the functionality described herein.

In other aspects, the disclosure encompasses at least one computer performing one or more method steps as described herein.

### Structural Overview

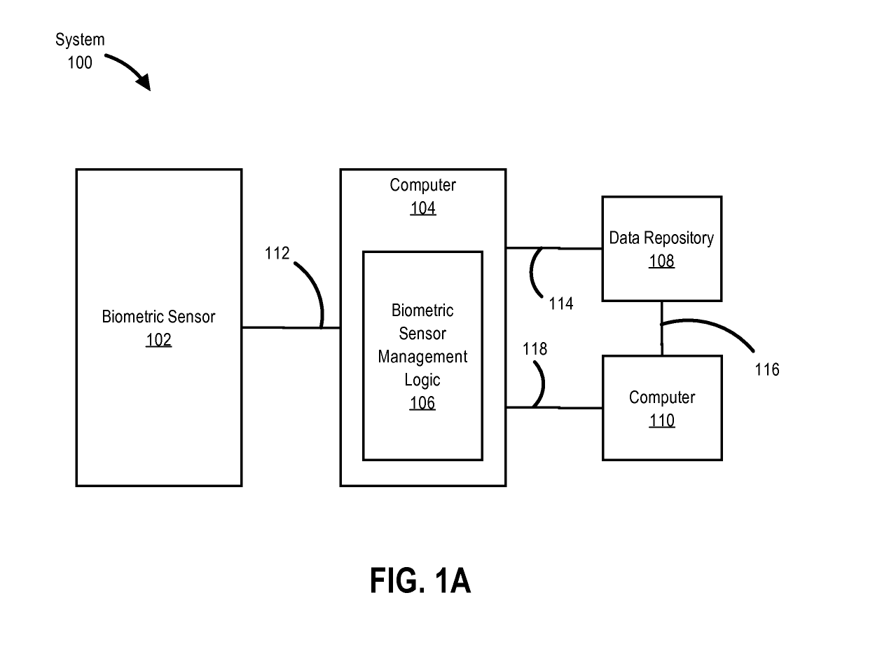

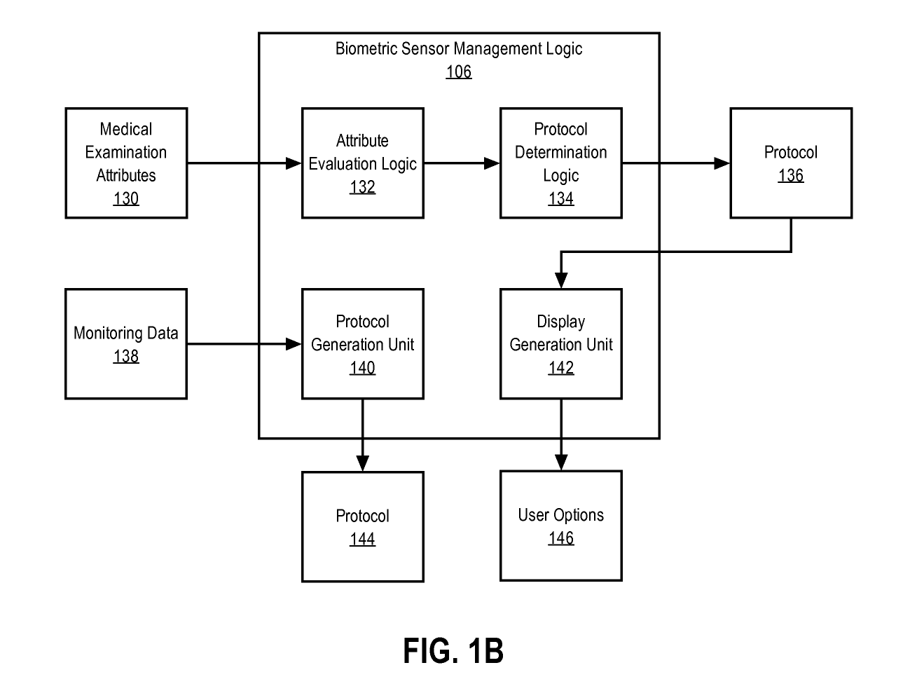

FIG. 1A illustrates a system in accordance with an embodiment. Although a specific system is described, other embodiments are applicable to any system that can be used to perform the functionality described herein. FIG. 1A illustrates a hypothetical system 100. Components of the system 100 may be connected by, without limitation, a network such as a Local Area Network (LAN), Wide Area Network (WAN), the Internet, Intranet, Extranet with terrestrial, satellite or wireless links, etc. Thus, in an embodiment, links 112, 114, 116, and 118 may each comprise a network link or cable. Alternatively or additionally, any number of devices connected within the network may also be directly connected to each other through wired or wireless communication segments.

In an embodiment, the system 100 includes one or more biometric sensors (e.g., biometric sensor 102), one or more computers (e.g., computer 104 and computer 110), and one or more data repositories (e.g., data repository 108). One or more components described within system 100 may be combined together in a single device. For example, biometric sensor 102 may be integrated with computer 104; and computer 110 and data repository 108 may be remotely coupled with computer 104 through one or more networks.

In an embodiment, the biometric sensor 102 generally represents any sensor used to obtain data related to a patient, which may be referred to herein as patient data. Patient data may include, without limitation, raw data obtained from a patient, an analysis of the patient data, textual information based on raw data, or images based on the raw data. The biometric sensor 102 may obtain patient data, for example, within a particular range from the patient, in direct contact with the patient, or applied to the patient through a conductive medium (e.g., gel).

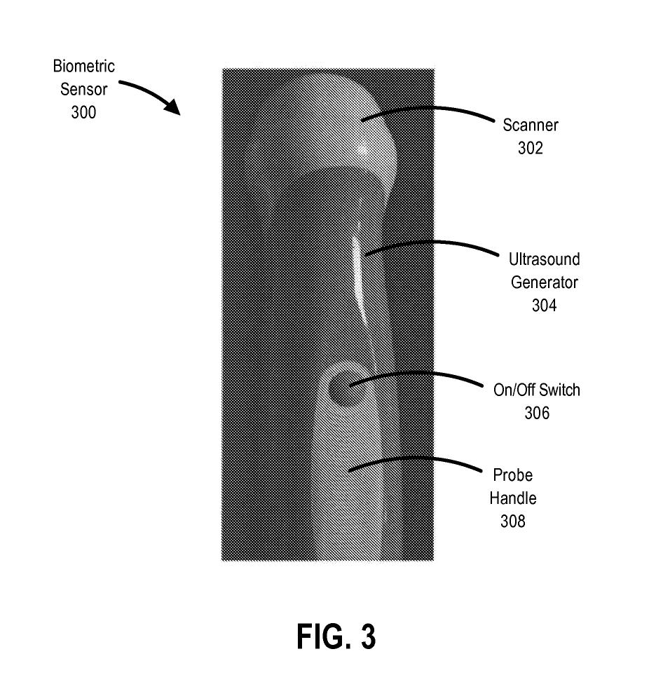

A biometric sensor 102 may refer to, for example, an ultrasound probe which obtains patient data through sound waves (e.g., with a frequency of 3.5 MHz, 5 MHz, 7.5 MHz, 12 MHz, etc.). FIG. 3 illustrates an ultrasound probe 300 as an example of a biometric sensor 102. An ultrasound probe may include a scanner 302, an ultrasound generator 304 to generate sound waves that are applied toward a patient through a gel or other conductive medium, an on/off switch 306, and a probe handle 308. An ultrasound probe may further include a receiver for capturing sound wave echoes which are used to generate image data to visualize subcutaneous body structures (e.g., tendons, muscles, joints, vessels, internal organs, fetuses in pregnant women). A biometric sensor 102 may be a handheld device which is operated by an operator. An operator may be a human or robotic operator that operates the biometric sensor 102 and/or the computer 104. Operating the biometric sensor 102 may include, without limitation, selecting device settings, configuring the device settings, and handling the biometric sensor 102 during a medical examination. Other examples of biometric sensors include, without limitation, medical cameras, electrocardiogram sensors, pulse oxymeters, and blood glucose monitors.

In an embodiment, a biometric sensor 102 may include a monitoring component for monitoring the use of the biometric sensor 102. The monitoring component may be located on the biometric sensor 102 itself or on another device (e.g., computer 104 or computer 110). The monitoring component may monitor, for example, device settings, device positions, device effectiveness, device use, etc.

In an embodiment, the biometric sensor 102 may be used to obtain patient data according to a protocol. A protocol generally represents directions for any procedure performed by an operator of the biometric sensor 102. A protocol may be represented in data stored in computer storage. A protocol may define organs that are to be probed and/or measured, actions that are to be performed by an operator, biometric sensor settings (e.g., gain control, intensity, contrast, depth, etc.), locations on a patient where the biometric sensor 102 is to be placed, etc. In an embodiment, a protocol may define specific data that is to be obtained (e.g., data for diagnosis of a particular medical condition) or a specific type of data (e.g., video data, frequency echoes, etc.).

In an embodiment, a protocol may include one or more steps taken be a human operator or machine operator to perform a medical examination. In an embodiment, a protocol may include one or more positions for placement of the biometric sensor 102. The positions for placement of the biometric sensor may be part of a chronological sequence. A protocol may include timing information such as a time period for applying pressure to a particular portion of the body to test for a medical condition.

In an embodiment, a protocol may include configuration settings (or other settings) for the biometric sensor 102, the computer 104, or another device within the system 100. For example, the protocol may include measurements of gain control, intensity, contrast, depth, etc. to be used for configuring the biometric sensor 102. The protocol may define attachments for the biometric sensor 102 to obtain particular patient data.

In an embodiment, each protocol may correspond to one or more medical examinations. For example, a protocol may define a particular procedure to test for symptoms or identify indications related to a particular disease or medical condition.

Protocols may be associated with particular patient types. For example, thin patients may require a different protocol than obese patients in order to obtain useful patient data. A first protocol may be defined for use with obese patients and a second protocol may be defined for use with thin patients. Patient types associated with differing corresponding protocols may be based, for example, on age, genetic characteristics, nationality, medical conditions, etc.

In an embodiment, computer 104 generally represents any device that includes a processor and is communicatively coupled with the biometric sensor 102. Examples of computer 104 include, without limitation, a desktop, a laptop, a tablet, a cellular phone, a smart phone, a personal digital assistant, a kiosk, etc. Computer 104 may be communicatively coupled with the biometric sensor 102 with wired and/or wireless segments. Computer 104 may be connected directly with the biometric sensor 102 using a universal serial bus (USB) cable.

Computer 104 may be used for determining, generating, or receiving one or more protocols for use with the biometric sensor 102 to obtain patient data as described below with respect to FIG. 1B. In an embodiment, computer 104 includes a biometric sensor management logic 106, which may comprise firmware, hardware, software, or a combination thereof in various embodiments that can implement the functions described herein. The computer 104 may include an interface component for displaying options to a user. For example, the computer 104 may include a display device configured to display available options related to the use of the biometric sensor 102.

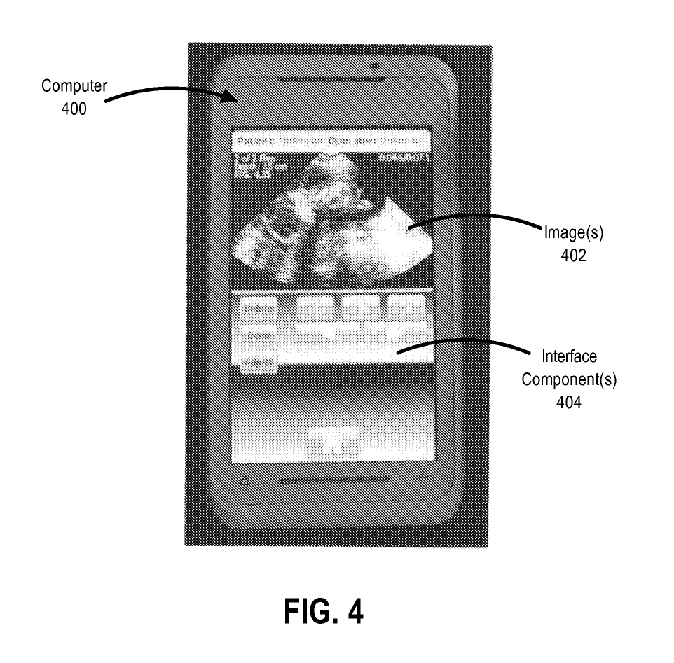

FIG. 4 illustrates a computer 400, as an example of computer 104, that may be used with a biometric sensor 102 such as an ultrasound probe. In an embodiment, computer 400 may include one or more buffers for temporarily or permanently recording patient data. For example, computer 400 may include functionality to display images 402 (or any other patient information) based on the patient data obtained by the biometric sensor 102. Data recorded in any buffer within computer 400 may be sampled at varying rates and using varying techniques. For example, every other image within a buffer may be sampled and transmitted to another computer (e.g., computer 110). In another example, every other horizontal vector or vertical vector from each image may be sampled and transmitted. A portion of interest of each image may be selected and transmitted. Different buffers within computer 400 may record the same patient data with varying levels of quality. For example, a particular buffer may include all patient data and another buffer may include a portion (e.g., based on sampling rate) of the patient data.

In an embodiment, the computer 400 may include logic configured to evaluate one or more medical examination attributes for determining one or more protocols. For example, the computer 400 may include components for querying a database based on one or more medical examination attributes and determining a protocol mapped to the one or more medical examination attributes. Medical examination attributes are further described below in relation to FIG. 1B.

In an embodiment, computer 400 may include logic configured to record data indicating the use of the biometric sensor 102. For example, the logic may include components configured for periodically receiving information about the placement of the biometric sensor 102 or configuration settings of the biometric sensor 102 used for obtaining patient data.

In an embodiment, computer 400 may include one or more interface components 404 to add, edit, delete, display, or send data accessible to the computer 400. In an embodiment, interface components 404 may be used to transmit one or more protocols to computer 110. The interface components 404 may be used to manage the biometric sensor 102.

In an embodiment, the data repository 108 generally represents any data storage device known in the art which may be configured to store data. Examples include local memory on computer 104, local memory on computer 110, shared memory, multiple servers connected over the internet, systems within a local area network, a memory on a mobile device, etc. In one or more embodiments, access to the data repository 108 may be restricted and/or secured. Access to the data repository 108 may require authentication using passwords, secret questions, personal identification numbers (PINs), and/or any other suitable authentication mechanism. Portions of data stored in the data repository 108 may be distributed and stored in multiple data repositories (e.g., servers across the world).

In one or more embodiments, the data repository 108 includes flat, hierarchical, network based, relational, dimensional, object modeled, or data files structured otherwise. For example, data repository 108 may be maintained as a table of an SQL database. In addition, data in the data repository 108 may be verified against data stored in other repositories.

Computer 110 may be implemented as described herein in relation to computer 104. Computer 110 may be located remotely from biometric sensor 102 and computer 104. Computer 110 may obtain data obtained by the biometric sensor 108 directly from the biometric sensor 108 or via computer 104. Computer 110 may be operated by a remote user to provide instructions which are transmitted to computer 104. Computer 104 or computer 110 may be configured to determine or receive one or more protocols for operating the biometric sensor 108. Computer 104 or computer 110 may comprise an analysis workstation for evaluating patient data.

### Architectural and Functional Overview

FIG. 1B illustrates an example of a biometric sensor management logic 106 located on computer 104. In an embodiment, the biometric sensor management logic 106 comprises an attribute evaluation logic 132 coupled to a protocol determination logic 134, a protocol generation unit 140 and a display generation unit 142. One or more components of the biometric sensor management logic 106 may be located on a different computer (e.g., computer 110) that is communicatively coupled with computer 104. One or more components of the biometric sensor management logic 106 may not be implemented within a particular embodiment. Additional components, that are not shown, may be implemented to perform functionality described herein. Computer 104 may be a special-purpose computer.

In an embodiment, the attribute evaluation logic 132 may comprise hardware, firmware, or software configured to receive and evaluate medical examination attributes 130. Medical examination attributes 130 may include, without limitation, patient information, exam information, device information, operator information, etc.

In an embodiment, patient information in medical examination attributes 130 may include patient history, previously administered medical examinations, to be administered medical examinations, medical examination results, patient preferences, patient parameters, patient medical conditions, patient symptoms, etc. Patient information may include nationality, ethnicity, place of residence, place of work, socio-economic group, age, genetic characteristics, behavioral habits, lifestyle habits, etc. of a patient from whom the patient data is obtained.

Patient information may further include a patient condition when the patient data was obtained. For example, the patient information may indicate that the patient data was obtained after a twelve hour fast, after a five minute jog, etc. In an embodiment, patient information may include device settings previously used for or preferred by the patient. For example, patient information may include a configuration of an ultrasound probe that was most effective for obtaining patient data from the patient.

In an embodiment, exam information in medical examination attributes 130 may include any information associated with a medical exam that was administered to a patient, is being administered to a patient, or will be administered to a patient. Examples of exam information include, without limitation, a type of exam, an exam name, and a type of patient that the exam is given to (e.g., pregnant patients, terminally ill patients, etc.).

In an embodiment, operator information in medical examination attributes 130 may include any information associated with an operator performing a medical examination using the biometric sensor 102. Operator information may include any operator information that is relevant to administering a medical examination, operating the biometric sensor 102, or the operator's understanding of instructions for operating the biometric sensor 102. Examples of operator information include, without limitation, a skill level of the operator, an education level of the operator, a language understood by the operator, an availability of the operator, a learning ability of the operator, a physical ability of the operator, a physical limitation of the operator, and a patient review (or other review) of the operator's performance.

In an embodiment, the attribute evaluation logic 132 may include logic configured to evaluate medical examination attributes to determine relevant information for a medical examination. For example, the attribute evaluation logic 132 may determine what patient data needs to be obtained, what medical condition(s) needs to be tested for, what medical examinations can or should be performed for a particular patient or using a particular device. Attribute evaluation logic 132 is configured to provide such data to the protocol determination logic 134.

In an embodiment, the protocol determination logic 134 may comprise hardware, firmware, or software configured for determining one or more protocols (e.g., protocol 136). As described above, a protocol generally represents any directions for a procedure performed by an operator of the biometric sensor 102 for obtaining patient data via the biometric sensor 102. The protocol determination logic 134 may determine a protocol 136 for obtaining patient data that needs to be obtained.

The protocol determination logic 134 may determine a protocol for a particular type of medical examination that is to be administered to a patient. For example, the protocol determination logic 134 may determine a protocol which identifies a gallbladder as an organ that is prone to developing gallstones. The protocol may further include operating instructions for an operator of a biometric sensor such as an ultrasound probe to check a gallbladder for gallstones. The protocol 136 determined by the protocol determination logic 134 may be customized or personalized based on the patient, operator, medical examination, or a combination thereof. The protocol determination logic 134 may be configured to provide protocol 136 to the display generation unit 142.

In an embodiment, the display generation unit 142 may comprise hardware, firmware, or software to generate interface components for display to a user. The display generation unit 142 may, for example, generate user options 146 that are based on one or more available protocols (e.g., protocol 136). The display generation unit 142 may include logic configured to select an applicable subset of data for display based on one or more protocols (e.g., protocol 136). The display generation unit 142 may be configured to display additional interface components for managing patient data, protocols, and/or the biometric sensor 102. The display generation unit 142 may be configured to analyze the biometric sensor 102 to determine available user options 146. For example, the display generation unit 142 may be configured to identify data collection attachments connected to the biometric sensor and select user options 146 that are applicable to the data collection attachments.

In an embodiment, the protocol generation unit 140 may comprise hardware, firmware, or software to generate a protocol 144. The protocol 144 may be similar in form or content to protocol 136 described above. The protocol generation unit 140 may include monitoring components for to obtain monitoring data 138 related to the use of the biometric sensor 102. The protocol generation unit 140 may include logic configured to periodically poll or request the biometric sensor 102 for obtaining information. For example, the protocol generation unit 140 may be configured to obtain position information from the biometric sensor 102. In an embodiment, the protocol generation unit 140 may interact with other components within computer 104 to obtain usage history for the biometric sensor 102.

All components of the biometric sensor management logic 106 may be integrated into a single unit of software, firmware, or a combination thereof in various embodiments. Thus, the separate blocks shown in FIG. 1B are provided solely to illustrate one example.

### Protocol Determination Using Medical Examination Attributes

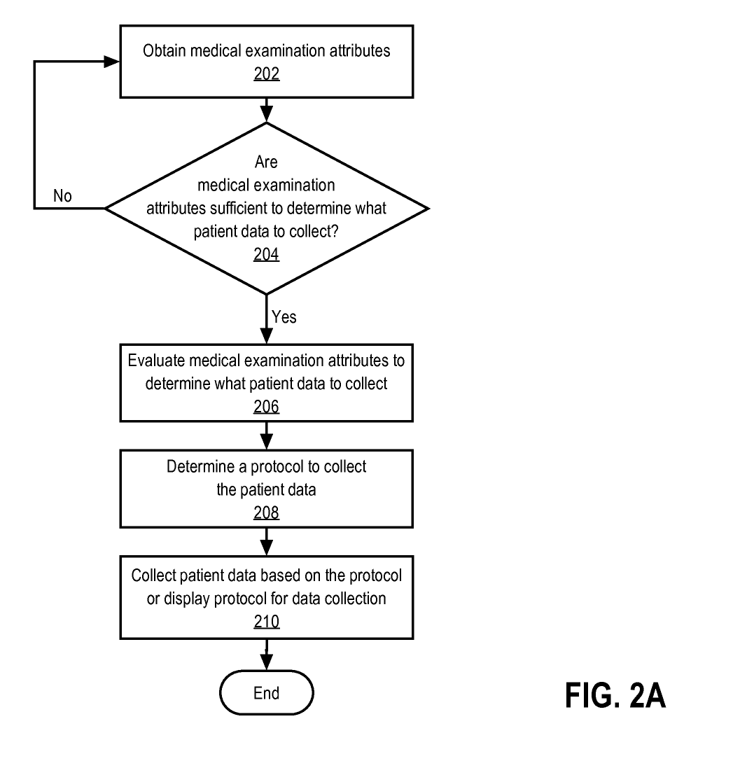

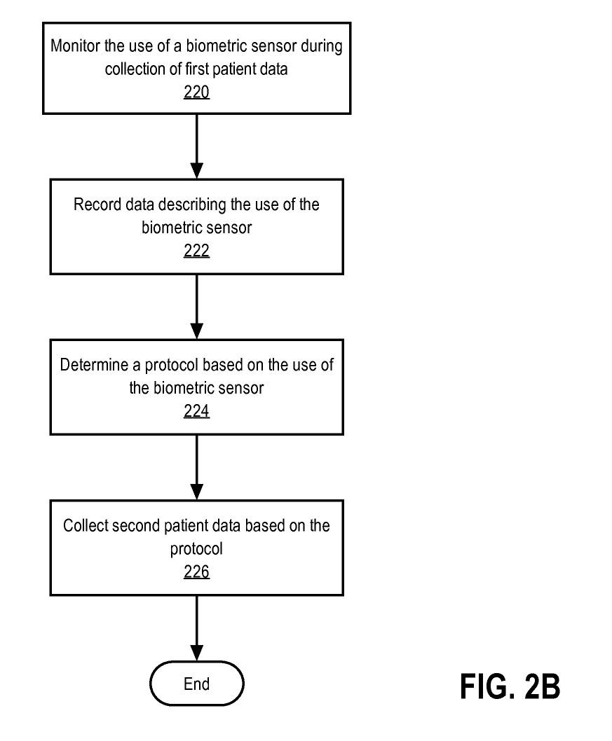

FIG. 2A illustrates determining a protocol for use with one or more biometric sensors. FIG. 2A may represent an algorithm that may be embodied in or hosted by the biometric sensor management logic 106. In an embodiment, one or more of the steps described below may be omitted, repeated, or performed in a different order. The specific arrangement shown in FIG. 2A is not required.

In Step 202, one or more medical examination attributes are obtained. The medical examination attributes may be obtained from a user. For example, a user may enter the medical examination attributes on computer 104. A user may enter the medical examination attributes on computer 110 for transmission to computer 104. In an embodiment, the medical examination attributes may be obtained or determined by computer 104. For example, a patient or operator may be prompted by computer 104 to place a finger on a scanner in order for the scanner to obtain a fingerprint. A database may then be queried to obtain the patient information or operator information based on the fingerprint. Querying a database may involve querying files stored locally on computer 104 or stored remotely on computer 110. In an embodiment, operator information may be obtained based on log in credentials provided, at computer 104, by an operator of the biometric sensor 102.

In Step 204, a determination may be made whether the medical examination attributes are sufficient to determine what patient data is to be obtained. For example, the patient information may indicate that in a previous examination of a pregnant patient, a 9-week exam was administered to the patient and that the previous examination was administered approximately four weeks prior to a current time. The patient information may further indicate that during an initial pregnancy period, a first exam is administered during the 9^{th} week of pregnancy and a second exam is administered during the 13^{th} week of pregnancy. This patient information may be used to determine that patient data is to be obtained from the patient for the 13-week exam.

In an embodiment, determining whether the medical examination attributes are sufficient to determine what patient data is to be obtained may be based on a number of applicable medical examinations. For example, based on one or more of patient information, operator information, and examination information, a number of suitable medical examinations may be identified. If the number of suitable medical examinations is under a specified threshold (e.g., 1 exam, 5 exams, 10 exams), then the sufficiency test in Step 204 may be passed.

In Step 205, the medical examination attributes are evaluated to determine what patient data to obtain. If patient information for a particular patient is submitted by a user, the patient information may be evaluated to identify scheduled examinations for that patient. In an example, operator information, for an operator of a biometric sensor 102, may be evaluated to determine medical exams which the operator is capable (e.g., trained, certified, etc.) of administering.

In Step 208, a protocol is determined for obtaining the patient data. In an embodiment, determining the protocol may include querying a database for instructions on obtaining the patient data. For example, the data repository 108 may be queried with 13-week pregnancy examination that is to be given to a pregnant patient. The results of a query may be used to select a subgroup of one or more available protocols as applicable protocols for the medical examination attributes.

In an embodiment, determining the protocol may include translating instructions to a different language. For example, patient information indicating that an operator speaks and understands Hindi may be used for determining that English-language operator instructions for performing a medical examination should be translated to Hindi-language operator instructions.

In an embodiment, determining the protocol may include selecting a complexity level for operator instructions. For example, based on the training, experience, or skill level of an operator, the protocol may include very detailed step-by-step instructions for each procedure or a list of procedures to be performed.

In an embodiment, determining a protocol may include selecting specific biometric sensor settings that are most suitable for the patient, operator, or medical exam. For example, determining the protocol may include determining a depth or intensity for an ultrasound probe based on the body mass index of a patient to obtain useful data. In an example, determining the protocol may include identifying attachments to the biometric sensor 102 that are suitable for obtaining the patient data that is to be obtained.

In an embodiment, determining a protocol may include determining external components that are used with the biometric sensor 102. The protocol may specify particular requirements for computer 104 (e.g., display resolution requirements, processing power requirements, size requirements, etc.) that is to be used with the biometric sensor 102 to obtain the patient data. For example, if an operator of a biometric sensor has poor vision, the protocol may specify a particular zoom level for a display screen and size of a display screen for the operator to obtain and view useful patient data. In an example, determining the protocol may involve determining a medication, lotion, gel, or other substance to be given to or applied to a patient. For a patient suffering from emphysema, a medical examination may be performed using a pulse oxymeter to determine if walking reduces the oxygen level in the patient's blood supply. The protocol, to perform a medical examination on a patient suffering from emphysema, may include instructions for the patient to walk and instructions on how the operator can use the pulse oxymeter to measure the oxygen level in the patient's blood subsequent to the patient walking.

In Step 210, the patient data may be obtained, based on the protocol, by one or more biometric sensors 102 or the computer may display the protocol for data collection. The patient data may be obtained by the biometric sensor 102 and transmitted to local computer 104 or remote computer 110. The obtained patient data may be aggregated with previously obtained patient data. For example, results of medical examinations for a particular patient may be charted over time to identify trends for the patient which may indicate an improvement or deterioration in health. In an embodiment, previously-obtained patient data and newly-obtained patient data may be used to identify further patient data that should be obtained to test for medical conditions indicated by a trend.

In an embodiment, multiple protocols determined in Step 206 may be presented to a user for selection. For example, patient history for a patient with multiple symptoms may be submitted to computer 104. Computer 104 may then evaluate the patient history to determine two or more protocols corresponding to different medical examinations related to the patient's symptoms. The two or more protocols may be presented to an operator. The protocols may be presented with descriptive information such as, for example, indicating what type of patient data is obtained using the protocol and what medical examination is given using the protocol. The operator, patient, or other user may then select one of the protocols.

In an embodiment, a selection of one of the possible protocols may be performed by computer 104. For example, the different protocols may evaluated in view of urgency or risk to patient and the most urgent protocol may be selected. In another example, the protocols may be evaluated in view of costs to a patient and a patient budget. Protocols with costs under the patient budget may then be selected. In response to a selection of a protocol, further details for that protocol may be provided to an operator for performing the medical examination.

In an embodiment, determining a protocol on medical examination attributes may be useful to customize and personalize each medical examination.

In an embodiment, determining a protocol based on medical examination attributes such as patient information may result in highly accurate protocols that obtain the most relevant patient data.

In an embodiment, determining a protocol based on medical examination attributes such as operator information may result in customizing instructions for an operator based on operator skill level, operator knowledge, operator experience, etc.

In an embodiment, determining a protocol based on medical examination attributes such as exam type information may be useful for ensuring that the protocol is correctly focused on the relevant patient data.

In an embodiment, determining a protocol based on medical examination attributes reduces the number of choices that an operator is required to make, which may result in a reduction of operator errors.

In an embodiment, determining a protocol based on medical examination attributes such as patient preferences or patient parameters may reduce the time needed for performing medical examinations.

### Protocol Determination Using Medical Examination Monitoring

FIG. 2B illustrates determining a protocol for use with one or more biometric sensors. FIG. 2B may represent an algorithm that may be embodied in or hosted by the biometric sensor management logic 106. In an embodiment, one or more of the steps described below may be omitted, repeated, or performed in a different order. The specific arrangement shown in FIG. 2B is not required.

In Step 220, the computer 104 may monitor the use of a biometric sensor 102 during collection of first patient data to obtain monitoring data. Monitoring the use of the biometric sensor may include recording instructions that are provided to the biometric sensor 102 by the computer 104. For example, device settings such as gain control selected at computer 104 for operating the biometric sensor 102 may be recorded by computer 104. In an embodiment, information related to the use of the biometric sensor 102 may be submitted by an operator of the biometric sensor 102.

In an embodiment, monitoring the use of the biometric sensor 102 may include requesting operating information for the biometric sensor 102 from the biometric sensor 102. For example, information may be requested periodically (e.g., every millisecond, every second, etc.) and aggregated to determine a chronological sequence of actions performed using the biometric sensor 102.

In an embodiment, monitoring the use of the biometric sensor may include obtaining data obtained by an accelerometer installed on the biometric sensor 102. The data may include sensor positioning information, acceleration data, or other data used for determining how the biometric sensor 102 was handled by an operator.

In Step 222, the monitoring data obtained in Step 220 may be recorded. Recording the monitoring data may include storing the monitoring data locally on computer 104 or remotely on computer 110. Recording the monitoring data may include uploading the monitoring data to a web server or a peer-to-peer system. Recording monitoring data may include storing the usage of the biometric sensor 102 without storing any patient identification information.

In Step 224, a protocol may be determined based on the monitoring data. Determining the protocol may involve a direct use of the monitoring data recorded during a medical examination of a patient. Determining the protocol may include analyzing different versions of the monitoring data recorded during different medical examinations. For example, a collection of monitoring data related to detecting gallstones may be analyzed to determine the most common technique or a technique used by highest rated operators. The protocol may then be determined based on this technique.

In an embodiment, the protocol may be published. For example, the protocol may be shared between devices, uploaded to web servers, or otherwise distributed. Biometric sensors (or computers connected to the biometric sensors) that are communicatively coupled may dynamically share protocols that are found to be effective to identify medical conditions or diagnose a patient. For example, if a set of biometric sensors 102 are being used by a team in the northern province of Zambia to diagnose members of the Bemba tribe, an operator may determine that a particular protocol is effective for diagnosing the tropical disease bilharzia. The operator may record the protocol by initiating a recording session, performing the particular protocol during a medical examination, and then terminating the recording session. The monitored data obtained by recording the protocol may then be transmitted to other members of the team that are also diagnosing members of the Bemba tribe.

In Step 226, second patient data may be obtained, based on the protocol, by one or more biometric sensors 102 or the computer may display the protocol for data collection. The protocol may be used with a different biometric sensor communicatively coupled to computer 110. The protocol may be used with the same biometric sensor 102 and computer 110 to obtain patient data from a different patient or with a different operator of the biometric sensor 102.

In an embodiment, determining a protocol based on monitored data may be useful to train new operators of the biometric sensors. Determining a protocol based on monitored data may be useful for dynamically modifying a medical examination strategy.

In an embodiment, determining a protocol based on monitored data may be useful for accurately capturing all aspects of a medical examination.

In an embodiment, determining a protocol based on monitored data may be useful for analyzing results of a medical examination. For example, the results may be reviewed in view of the exact steps performed. This review may be used, for example, to determine whether the results are reliable and which body part the results relate to.

### Hardware Overview

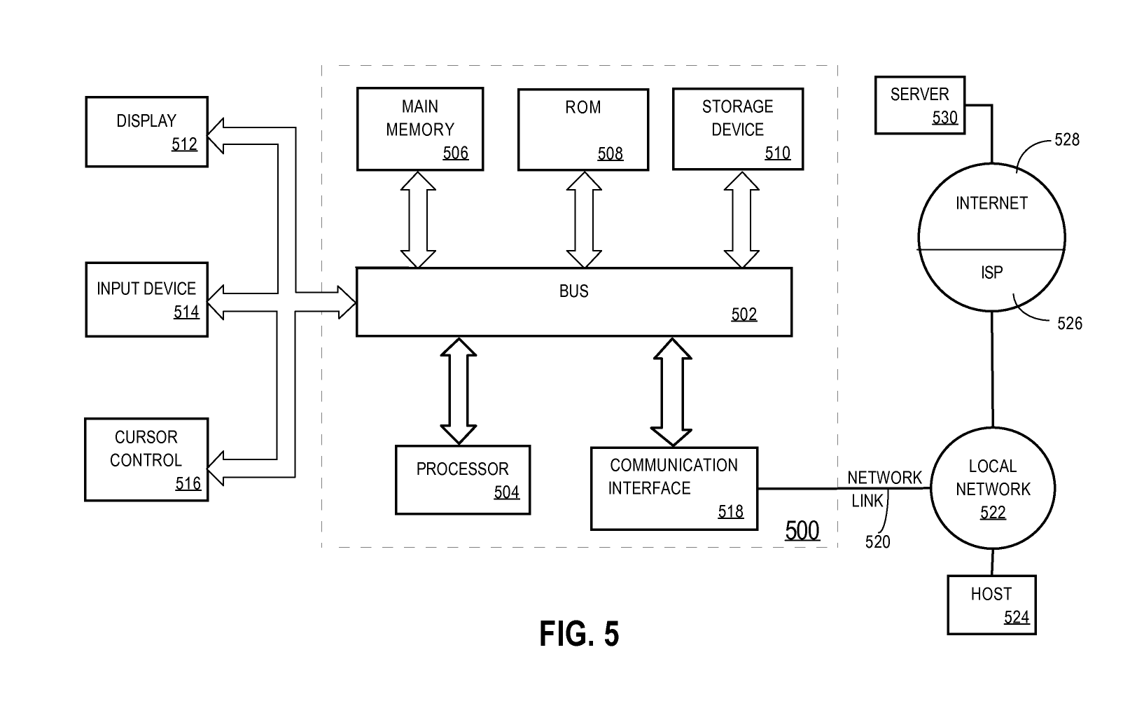

FIG. 5 is a block diagram that illustrates a computer system 500 upon which an embodiment may be implemented. Computer system 500 includes a bus 502 or other communication mechanism for communicating information, and a processor 504 coupled with bus 502 for processing information. Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

The invention is related to the use of computer system 500 for implementing the techniques described herein. According to one embodiment, those techniques are performed by computer system 500 in response to processor 504 executing one or more sequences of one or more instructions contained in main memory 506. Such instructions may be read into main memory 506 from another machine-readable medium, such as storage device 510. Execution of the sequences of instructions contained in main memory 506 causes processor 504 to perform the process steps described herein. In alternative embodiments, hard-wired circuitry may be used in place of or in combination with software instructions to implement the invention. Thus, embodiments are not limited to any specific combination of hardware circuitry and software.

The term “machine-readable medium” as used herein refers to any medium that participates in providing data that causes a machine to operation in a specific fashion. In an embodiment implemented using computer system 500, various machine-readable media are involved, for example, in providing instructions to processor 504 for execution. Such a medium may take many forms, including but not limited to storage media and transmission media. Storage media includes both non-volatile media and volatile media. Non-volatile media includes, for example, optical or magnetic disks, such as storage device 510. Volatile media includes dynamic memory, such as main memory 506. Transmission media includes coaxial cables, copper wire and fiber optics, including the wires that comprise bus 502. Transmission media can also take the form of acoustic or light waves, such as those generated during radio-wave and infra-red data communications. All such media must be tangible to enable the instructions carried by the media to be detected by a physical mechanism that reads the instructions into a machine.

Common forms of machine-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, and EPROM, a FLASH-EPROM, any other memory chip or cartridge, a carrier wave as described hereinafter, or any other medium from which a computer can read.

Various forms of machine-readable media may be involved in carrying one or more sequences of one or more instructions to processor 504 for execution. For example, the instructions may initially be carried on a magnetic disk of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are exemplary forms of carrier waves transporting the information.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution. In this manner, computer system 500 may obtain application code in the form of a carrier wave.

In the foregoing specification, embodiments have been described with reference to numerous specific details that may vary from implementation to implementation. Thus, the sole and exclusive indicator of what is the invention, and is intended by the applicants to be the invention, is the set of claims that issue from this application, in the specific form in which such claims issue, including any subsequent correction. Any definitions expressly set forth herein for terms contained in such claims shall govern the meaning of such terms as used in the claims. Hence, no limitation, element, property, feature, advantage or attribute that is not expressly recited in a claim should limit the scope of such claim in any way. The specification and drawings are, accordingly, to be regarded in an illustrative rather than a restrictive sense.

What is claimed is:

1. A method comprising:
obtaining, by a computer, a plurality of medical examination attributes comprising patient information and operator information;
determining, by the computer, a protocol for obtaining patient data from a patient based on the patient information and the operator information;
obtaining, by the one or more biometric sensors, the patient data from the patient based on the protocol.

2. The method as recited in claim 1, wherein the protocol comprises an ultrasound sensor probing sequence.

3. The method as recited in claim 1, wherein determining the protocol is further based on exam type information associated with a medical exam that could be performed on the patient to obtain the patient data.

4. The method as recited in claim 1, wherein the protocol comprises one or more of: a configuration setting for the one or more biometric sensors or positioning information for the one or more biometric sensors.

5. The method as recited in claim 1, further comprising:
determining, by the computer, a subgroup of one or more protocols from a plurality of protocols for obtaining patient data from the patient based on the patient information and the operator information;
presenting the subgroup of one or more protocols as options for use with the one or more biometric sensors.

6. The method as recited in claim 1, further comprising comparing, by the computer, a first patient data that is newly obtained by the one or more biometric sensors with a second patient data that was previously obtained by the one or more biometric sensors and stored on the computer, wherein the computer is communicatively coupled with the one or more biometric sensors.

7. A method comprising:
monitoring, by a computer, a biometric sensor obtaining first patient data from a first patient during a medical examination;
recording monitoring data comprising one or more configurations, of the biometric sensor, while the first patient data was being obtained;
generating a protocol for obtaining second patient data from a second patient based on the monitoring data;
wherein the method is performed by at least one computer.

8. The method as recited in claim 7, wherein the one or more configurations comprises one or more of:
a plurality of positions for the biometric sensor while the first patient data was being obtained;
a test sequence for obtaining the first patient data; or
device settings for the biometric sensor.

9. A computer comprising:
one or more processors;
an attribute evaluation logic coupled to the one or more processors and configured to obtain a plurality of medical examination attributes comprising patient information and operator information;
a protocol determination logic coupled to the one or more processors and configured to determine a protocol for obtaining patient data from a patient based on the patient information and the operator information;
wherein the computer is communicatively coupled with one or more biometric sensors configured to obtain the patient data from the patient based on the protocol.

10. The computer as recited in claim 9, wherein the protocol comprises an ultrasound sensor probing sequence.

11. The computer as recited in claim 9, wherein determining the protocol is further based on exam type information associated with a medical exam that could be performed on the patient to obtain the patient data.

12. The computer as recited in claim 9, wherein the protocol comprises one or more of: a configuration setting for the one or more biometric sensors or positioning information for the one or more biometric sensors.

13. The computer as recited in claim 9, further comprising:
determining, by the computer, a subgroup of one or more protocols from a plurality of protocols for obtaining patient data from the patient based on the patient information and the operator information;
presenting the subgroup of one or more protocols as options for use with the one or more biometric sensors.

14. The computer as recited in claim 9, further comprising a display generation unit for presenting a comparison of a first patient data and a second patient data obtained by the one or more biometric sensors from the patient at a first time and at a second time, respectively.

15. A computer comprising:
one or more processors;
a protocol generation unit coupled to the one or more processors and configured to:
monitor a biometric sensor obtaining first patient data from a first patient during a medical examination;
record monitoring data comprising one or more configurations, of the biometric sensor, while the first patient data was being obtained;
generate a protocol for obtaining second patient data from a second patient based on the monitoring data.

16. A computer as recited in claim 15, wherein the one or more configurations comprises one or more of:
a plurality of positions for the biometric sensor while the first patient data was being obtained;
a test sequence for obtaining the first patient data; or
device settings for the biometric sensor.

17. A non-transitory computer readable storage medium comprising instructions, which when executed by the one or more processors, cause:
obtaining, by a computer, a plurality of medical examination attributes comprising patient information and operator information;
determining, by the computer, a protocol for obtaining patient data from a patient based on the patient information and the operator information;
obtaining, by the one or more biometric sensors, the patient data from the patient based on the protocol.

18. The computer readable storage medium as recited in claim 17, wherein the protocol comprises an ultrasound sensor probing sequence.

19. The computer readable storage medium as recited in claim 17, wherein instructions for determining the protocol further comprise instructions for determining the protocol based on exam type information associated with a medical exam that could be performed on the patient to obtain the patient data.

20. The computer readable storage medium as recited in claim 17, wherein the protocol comprises one or more of: a configuration setting for the one or more biometric sensors or positioning information for the one or more biometric sensors.

21. The computer readable storage medium as recited in claim 17, further comprising instructions, which executed by the one or more processors, cause:
determining a subgroup of one or more protocols from a plurality of protocols for obtaining patient data from the patient based on the patient information and the operator information;
presenting the subgroup of one or more protocols as options for use with the one or more biometric sensors.

22. The computer readable storage medium as recited in claim 17, further comprising instructions, which executed by the one or more processors, cause comparing a first patient data that is newly obtained by the one or more biometric sensors with a second patient data that was previously obtained by the one or more biometric sensors and stored on a computer communicatively coupled with the one or more biometric sensors.

23. A non-transitory computer readable storage medium comprising instructions, which when executed by the one or more processors, cause:
monitoring, by a computer, a biometric sensor obtaining first patient data from a first patient during a medical examination;
recording monitoring data comprising one or more configurations, of the biometric sensor, while the first patient data was being obtained;
generating a protocol for obtaining second patient data from a second patient based on the monitoring data;
wherein the method is performed by at least one computer.

24. The computer readable storage medium as recited in claim 23, wherein the one or more configurations comprises one or more of:
a plurality of positions for the biometric sensor while the first patient data was being obtained;
a test sequence for obtaining the first patient data; or
device settings for the biometric sensor.
